(19) United States
(12) Patent Application Publication
(10) Pub. No.: US 2015/0141763 A1
(43) Pub. Date: May 21, 2015

(54) NON-INVASIVE BLOOD GLUCOSE CONCENTRATION SENSING USING LIGHT MODULATION

(71) Applicant: HONEYWELL INTERNATIONAL INC., Morristown, NJ (US)

(72) Inventors: Hans Roth, Phoenix, AZ (US); Santosh Mathan, Seattle, WA (US)

(73) Assignee: HONEYWELL INTERNATIONAL INC., Morristown, NJ (US)

(21) Appl. No.: 14/206,520

(22) Filed: Mar. 12, 2014

Related U.S. Application Data
(60) Provisional application No. 61/905,400, filed on Nov. 18, 2013.

(51) Int. Cl. A61B 5/145; A61B 5/0484; A61B 5/0496; A61B 5/04; A61B 5/0482; A61B 5/1455; A61B 5/00

(52) U.S. Cl. CPC A61B 5/14532; A61B 5/1455; A61B 5/04842; A61B 5/7246; A61B 5/04012; A61B 5/746; A61B 5/0482; A61B 5/0496
USPC 600/301

(57) ABSTRACT

A non-invasive blood glucose concentration sensing system and method includes emitting light of a first color into an eye of the user and then emitting light of a second color into the eye of the user, or flashing blue light into the eye of the user. Neurophysiological brain activity and electrical responses of the eye of the user are sensed during and after emitting or flashing of the light into the eye of the user. In a processor, one or both of the sensed neurophysiological brain activity and the sensed electrical responses of the eye are correlated to the glucose concentration in the blood of the user.

### CROSS-REFERENCE TO RELATED APPLICATION

This application claims the benefit of U.S. Provisional Application No. 61/905,400, filed Nov. 18, 2013.

### TECHNICAL FIELD

The present invention generally relates to blood glucose concentration measurement, and more particularly to a non-invasive blood glucose concentration sensing and method that uses light modulation.

### BACKGROUND

Approximately 380 million people worldwide have diabetes today; that number is projected to grow to 600 million by the year 2035. It is estimated that by the year 2016, upwards of sixteen billion dollars ($16B) will be spent annually on blood glucose diagnostics and testing. This may be an inaccurately low estimate since many diabetics (and pre-diabetics), for various reasons, are unwilling to test themselves. One of the most prevalent reasons for not testing relates the self-testing methodologies, which have advanced relatively little over the past 30 years. Most methodologies continue to rely on finger pricking that can cause pain, discomfort, and inconvenience for users. Importantly, the human and financial costs of diabetes may be reduced through more facile testing methods.

In recent years, efforts have been made to implement and bring to market various types of sufficiently accurate, non-invasive blood glucose concentration testing methods. Some of these methods include passing light waves through solid tissues, such as a fingertip or an ear lobe, and measuring the molecular absorption spectrum of glucose. Because of the variability of absorption and scatter of electromagnetic energy in solid tissue, these methods have been generally unsuccessful. Other methods include measuring blood glucose in various other body fluids, such as the anterior chamber, tears, and interstitial fluids. These methods have shown only limited success.

Another method that was once thought promising, centers around the discovery that the regeneration rate of visual pigment in the eyes depends strongly on the blood glucose concentration, and that visual pigment regeneration could be measured within seconds of bleaching the visual pigment in the retina of an eye. This methodology has thus far proved commercially unsuccessful due to temporal and idiosyncratic issues of measuring subtle changes in blood glucose concentration from direct measurement of pigment regeneration.

Hence, there is a need for a relatively simple, painless, non-invasive system and method for determining a person's blood glucose concentration. The present invention addresses at least this need.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

In one embodiment, a method for determining glucose concentration in the blood of a user includes emitting light of a first color into an eye of the user, and emitting light of a second color into the eye of the user. Neurophysiological brain activity and electrical responses of the eye of the user are sensed during and after emitting the light of the first and second colors into the eye of the user. In a processor, one or both of the sensed neurophysiological brain activity and the sensed electrical responses of the eye are correlated to the glucose concentration in the blood of the user.

In another embodiment, a non-invasive blood glucose concentration sensing system includes a light source, a neurophysiological brain activity sensor, an electroretinography (ERG) sensor, and a processor. The light source is configured to emit light of a first color and light of a second color into an eye of a user. The neurophysiological brain activity sensor is configured to sense neurophysiological brain activity of the user and supply neurophysiological brain activity signals representative thereof. The ERG sensor is configured to sense electrical responses of the eye of the user and supply ERG signals representative thereof. The processor is in operable communication with the light source, the neurophysiological brain activity sensor, and the ERG sensor. The processor is coupled to receive the neurophysiological brain activity signals and the ERG signals and is configured to control the light source to emit the light of the first color and then emit the light of the second color, process the neurophysiological brain activity signals and the ERG signals, and correlate one or both of the sensed neurophysiological brain activity and the sensed electrical responses of the eye to the glucose concentration in the blood of the user.

In yet another embodiment, a method for determining glucose concentration in the blood of a user includes emitting blue light into an eye of the user. Neurophysiological brain activity and electrical responses of the eye of the user are sensed during and after emitting the blue light is flashed into the eye of the user. One or both of the sensed neurophysiological brain activity and the sensed electrical responses of the eye are correlated to the glucose concentration in the blood of the user.

Furthermore, other desirable features and characteristics of the blood glucose concentration sensing system and methods will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and wherein:

FIG. 1 a functional block diagram of one embodiment of a non-invasive blood glucose concentration sensing system;

FIG. 2 is a representation of one particular physical implementation of the system of FIG. 1; and

FIGS. 3-5 depict different exemplary processes, in flowchart form, that the system of FIG. 1 may implement to determine the blood glucose concentration of a user.

### DETAILED DESCRIPTION

The following detailed description is merely exemplary in nature and is not intended to limit the invention or the application and uses of the invention. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention which is defined by the claims. Furthermore, there is no intention to be bound by any expressed or implied theory presented in the preceding technical field, background, brief summary, or the following detailed description.

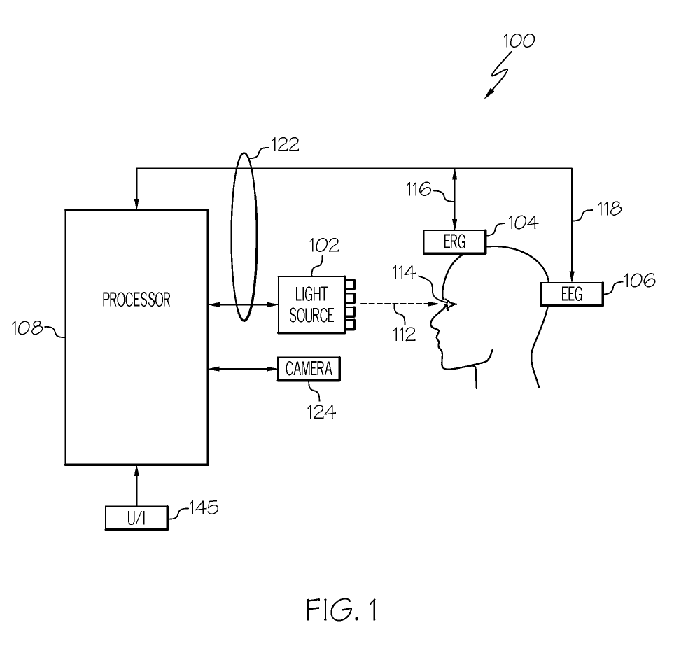

Referring to FIG. 1, a functional block diagram of one embodiment of a non-invasive blood glucose concentration sensing system 100 is depicted and includes a light source 102, an electroretinography (ERG) sensor 104, a neurophysiological brain activity sensor 106, and a processor 108. The light source 102 is in operable communication with the processor 108 and is configured to selectively emit light 112 into at least one eye 114 of a user 110. Although the light source 102 is depicted as emitting light 112 into only one eye 114, it will be appreciated that it could be configured to emit light 112 into both eyes 114. Depending upon the color and/or intensity, the light 112, when emitted into the eye(s) 114 of the user 110, will promote an evoked potential which may be translated into a signal of interest either through bleaching certain photoreceptors in the eye(s) 114, stimulating recovery of the photoreceptors from bleaching, damping and stimulating electrical processing in the visual centers of the brain, or various combination of these neurophysiological processes. Thus, the light source 102 is configured to emit light 112, at various time points, of various colors and/or intensities. The reason for this will be described in more detail further below. It will be appreciated that the light source 102 may be variously implemented, but in the depicted embodiment it is implemented using a plurality of light emitting diodes (LEDs), each of which is configured to emit a light of a different color. In a particular preferred embodiment, these colors include red, green, blue, yellow, and white.

The ERG sensor 104 is in operable communication with the processor 108. The ERG sensor 104 is configured to sense electrical responses of various photoreceptor cell types in the eye(s) 114 of the user 110, and supply ERG signals 116 representative thereof. In the embodiment depicted in FIG. 1, the ERG sensor 104 is configured to be disposed on, near, or otherwise coupled to, the user 110, and is implemented using one or more electrodes that are disposed on the eye(s) 114, the skin near the eye(s) 114 and/or near the skin as technology supports. The ERG signals 116 supplied by the ERG sensor 104 comprise electrical potentials from the different photoreceptor cell types within the eye(s) 114. These electrical potentials, generally known as visual evoked potentials (VEPs) are electrical potentials that are initiated by visual stimuli, such as the above-described.

The neurophysiological brain activity sensor 106 is configured to sense neurophysiological brain activity of the user 110, and to supply neurophysiological brain activity signals 118 representative thereof. In the embodiment depicted in FIG. 1, the neurophysiological brain activity sensor 106 is configured to be disposed on or near, or otherwise coupled to, the user 110, and is implemented using a single or plurality of electroencephalogram (EEG) sensors 106. The EEG sensors 106 are configured to be disposed on or near the head of the user 110. The neurophysiological brain activity sensor 106 may additionally be configured to sense various types of neurophysiological brain activity, and thus supply various types of neurophysiological brain activity signals 118. Preferably, however, the neurophysiological brain activity sensor 106 is configured to sense brain activity and VEPs that are initiated by visual stimuli, such as the above-described.

The processor 108 is in operable communication with the light source 102, the ERG sensor 104, if included, the neurophysiological brain activity sensor 106 via, for example, direct contact from the device one or more communication buses or cables 122 or via wireless communication. As was previously noted, the light source 102 is configured to emit light 112 of various colors and/or intensities, and at varying time sequences. In some embodiments, the processor 108 is configured to control the light source 102 to emit a first color of light 112 and then emit a second color of light. The processor 108 may be configured to emit the first color of light at a gradually increasing intensity or graduated rate for a predetermined time period to attain a predetermined maximum intensity, and then emit a second color of light at a gradually increasing intensity or a graduated rate for the same or different predetermined time period to attain the same or different predetermined maximum intensity. Alternatively, the processor 108 may be configured to emit the first color of light as a pulse or flash for a predetermined time period and at a predetermined intensity, and then emit the second color of light as a pulse or flash for the same time or different period and the same predetermined or different intensity. In still other embodiments, the light of both the first and second colors may be emitted as pulses or flashes that vary in intensity and emission time. The variation in intensity may occur within each individual pulse or flash, between different pulses or flashes, or in any other sequence or form useful. The variation in emission time may also occur within each individual pulse or flash, between different pulses or flashes, or both. Moreover, the first and second colors may vary, but in one particular embodiment the first and second colors are what are generally referred to as “opposing colors.” Such opposing colors include red and green, blue and yellow, and white and black.

The above-described light source control scheme is referred to herein as “color modulation” or “color frequency modulation.” In one particular embodiment, the color modulation is from red-to-green, blue-to-yellow, or white-to-black. The particular color modulation scheme that is used will depend upon the photoreceptors being stimulated. As is generally known, the red/green pair, the blue/yellow pair and the white/black are opponent. Moreover, if it is desired to stimulate the intrinsically photosensitive retinal ganglion cells (ipRGCs), then blue/blue, blue/green or blue/yellow color modulation may be used. In some instances, it may be desirable to use just a single blue pulse, flash or graduation of light 112.

In one embodiment, the processor 108 is configured to control the light source 102 such that pigment bleaching is limited to a predetermined maximum bleach percentage, and/or predetermined intensity of light for a predetermined period of time, and then modulate the color frequency. The range of bleaching percentage may vary, but in one particular embodiment it is a range of about 0-20%. Greater percentages of bleaching are, of course, possible, but it can take a relatively long time for the eye(s) 114 to recover therefrom. With this exemplary color modulation scheme, the processor 108 will control the light source 102 to emit light at a frequency that corresponds with long-preferring cones (e.g., red) and at a gradually increasing intensity to a steady-state until the predetermined maximum bleach is achieved. Thereafter, the processor 108 will control the light source 102 to emit light at a frequency corresponding with middle-preferring cones (e.g., green).

The peak light intensities and/or time to reach the peak light intensities during the color modulation process may vary from user-to-user. Indeed, these parameters, as well as the analysis of VEP signals, may be based on individual baseline data (ERG/EEG, heart rate and other parametric data), user profiles, historic data, environmental data, sensor data (including photometric), and/or user provided/input data. In addition, these parameters may also vary based on an instantaneous evaluation of retinal health. Such evaluation may be facilitated via a camera 124, which may also be associated with this system 100. The camera 124 will allow the user 110 to take a picture of their eye(s) 114. The processor 108, using imagery from the camera 124 and known processing techniques, may evaluate the health of the retina and be used to predetermine the light intensity, peaks, waveforms and times. The processor 108 may also (or instead) evaluate the health of the retina and predetermine the light intensity, peaks, waveforms and times from either or both of the ERG signals 116 and EEG signals 118.

The processor 108, in addition to controlling the light source 102, is coupled to receive the ERG signals 116 from the ERG sensor 104 and, if included, the neurophysiological brain activity signals 118 from the neurophysiological brain activity sensor 106. The processor 108 is configured, upon receipt of these signals 116, 118, and upon analysis of other above-described data inputs, to correlate the sensed electrical response of the eye(s) 114 and/or the sensed neurophysiological brain activity to the blood glucose concentration of the user 110. In particular, prior to and throughout the color modulation process (e.g., red-to-green, blue-to-yellow, white-to-black, and/or blue-to-blue, blue-to-green and blue only), the ERG signals 116 and neurophysiological brain activity signals 118 are retrieved and one or both are analyzed by the processor 108, either without or in conjunction with analysis of the other above-described data inputs, to determine the rate and extent of visual pigment regeneration and/or the corresponding brain activity, and thus blood glucose concentration. These data may also be stored in a memory buffer, a database, or the like. Historical data may facilitate trending useful in long-term glucose management (and may offer insight into individual disease progressions, for example early historical information may support evaluation of insulin sensitivity, sugar processing and more).

More specifically, it is generally known that the rate and extent of visual pigment regeneration can be correlated to blood glucose concentrations. It will be appreciated that the rate and extent of visual pigment regeneration may be determined from the VEPs sensed by the ERG sensor 106, the EEG sensors 106, or both. It has also been shown that the initiation of VEPs within photoreceptor cells is a function of blood glucose concentrations. With regard to analysis of the ERG and EEG signals 116, 118, changes in the amplitude and latency of specific peaks of each of these signals are measured, and changes in peak latency and peak amplitudes therein are used to evaluate visual pigment regeneration and initiation of VEPs. The time course of changes to peak latency and amplitude measures serve as measures of regeneration and initiation, or in the case of ipRGCs as shifts from baseline potentials. These data could be used in several ways. For example, the data could be compared against group norms, or to an individualized baseline created in conjunction with manipulation of blood sugar levels.

It is noted that ipRGCs do not bleach similar to other photoreceptors (i.e., rods and cones), but like these other photoreceptors are dependent upon blood glucose energy to initiate electrical potentials. As such, ipRGCs can also be used to determine blood glucose concentration. To do so, the processor 108 determines the onset of signal, for example, and peak latencies and amplitudes in conjunction with or without other data inputs described herein. Moreover, an interesting characteristic of ipRGCs is that these photoreceptors trigger ERG/EEG responses in individuals with compromised eyesight. These data may be used in several ways. For example, the data could be compared against group norms, or to an individualized baseline created in conjunction with manipulation of blood sugar levels.

The system may also include a user-interface 145 to allow the user 110 to input various parameters to support the investigation of the underlying contributors to disease onset and progression or baseline variation. The parameters may vary and may include, for example, dietary information, time, location, sensor inputs (e.g., photometric and other) and other environmental/individual parameters.

In some embodiments, the system 100 may be configured to provide continuous (e.g., “24/7”) monitoring of blood glucose concentration. In these embodiments, the processor 108 is additionally configured to control the light source 102 to emit only certain light frequencies during sleep hours. For example, the processor 108 may control the light source 102 to emit only red light or red and yellow light, since these frequencies minimally impact circadian sleep patterns and are generally know to pass through the eyelid producing measurable VEPs. If, during sleep, the system 100 determines that the user's blood glucose concentration is at a predetermined high or low concentration, the processor 108 may also control the light source 102 to emit light, such as blue or green, as stimuli to slowly or more abruptly wake a user. In particular, short bursts of blue and/or green light of relatively high intensity, or pre-determined graduations of light will stimulate wakefulness and cognition.

The system 100 may also be configured to generate one or more alerts and/or to generate various types of feedback. The alerts, which may be implemented audibly, visually, haptically, or by various combinations of these methods, may be used to remind the user 110 to check their blood glucose concentration. The alerts may be rendered on a display device, supplied via an audio device, delivered by a mechanical, acoustic, or other means, or transmitted to a remote device as an email or a text message. The feedback may also be implemented audibly, visually, haptically, or by various combinations of these methods, and is representative of the glucose concentration in the blood of the user. Similar to the alerts, the feedback may be rendered on a display device, supplied via an audio device, delivered by a mechanical, acoustic, or other means, or transmitted to a remote device as an email or a text message.

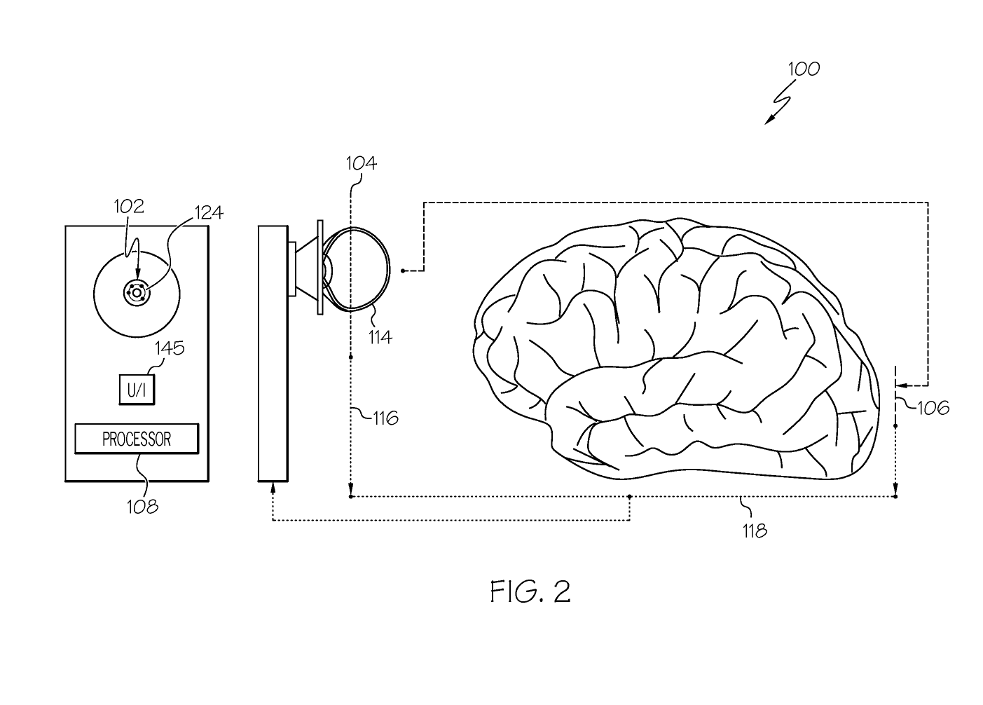

It will be appreciated that the light source 102, the processor 108, and camera 124 (if included) may, in some embodiments, be packaged together in a hand-held device, such as the one depicted in FIG. 2. In particular, these elements could be implemented in a smart phone, a tablet computer, a near-to-eye device or various other hand-held or wearable devices. These elements could also be implemented into a device, such as a sleeping mask or glasses frame, to be worn by a user during sleep hours or throughout the day.

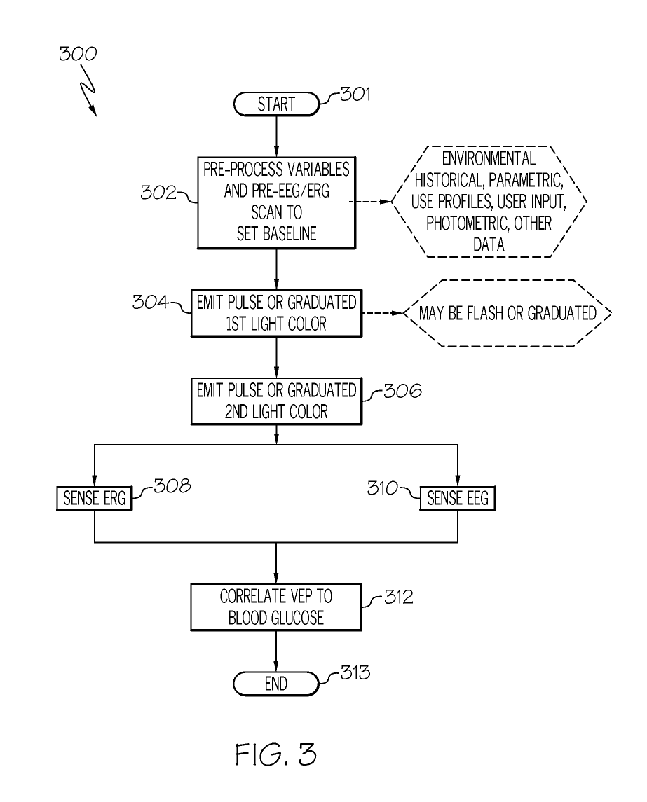

It was noted above that the processor 108 controls the emission of the light 112 from the light source 102 and, upon receipt of the ERG signals 116 and the neurophysiological brain activity signals 118, correlates the sensed electrical responses of the eye(s) 114 the sensed neurophysiological brain activity to the blood glucose concentration of the user 110. It was additionally noted that the system 100 may be configured to provide continuous monitoring. The processes 200, 300 by which the processor 108 implements these functions are depicted in flowchart form in FIGS. 3-5, respectively. These processes 300, 400, 500 will now be described in more detail, beginning first with the process 300 depicted in FIG. 3. Before doing so, however, it is noted that the depicted processes 300, 400, 500 are merely exemplary of any one of numerous ways of depicting and implementing the overall processes to be described. It is additionally noted that the numerical parenthetical references in the following description refer to like blocks in the flowcharts depicted in FIGS. 3-5.

Turning now to the description of the process 300 depicted in FIG. 3, the processor 108 commands a baseline reading of the ERG and/or EEG sensors 104, 106 and pre-processes these readings and, in some embodiments, other pre-process variables to set a baseline (302). These other pre-process variables include, as noted above, various individual baseline data (heart rate and other parametric data), user profiles, historic data, environmental data, sensor data (including photometric), and/or user provided/input data. Thereafter, the processor 108 commands the light source 102 to first emit light 112 of a first color into the eye(s) 114 of the user 110 (304), and then to emit light 112 of a second color into the eye(s) 114 of the user 110 (306). The different color light 112, as noted above, bleach different photoreceptors in the eye(s) 114, causing the different photoreceptors to undergo pigment regeneration or causing the different photoreceptors to stimulate neurophysiologic activity from the initiation of VEPs. During the bleaching and pigment regeneration processes, or the stimulation of VEPs, the electrical responses of the eye(s) 114 are sensed by the ERG sensor 104 (308) and/or the neurophysiological brain activity is sensed by the neurophysiological brain activity sensors 106 (310). The processor 108 correlates the sensed electrical responses of the eye(s) 114 and the sensed neurophysiological brain activity to the glucose concentration in the blood of the user 110 (312).

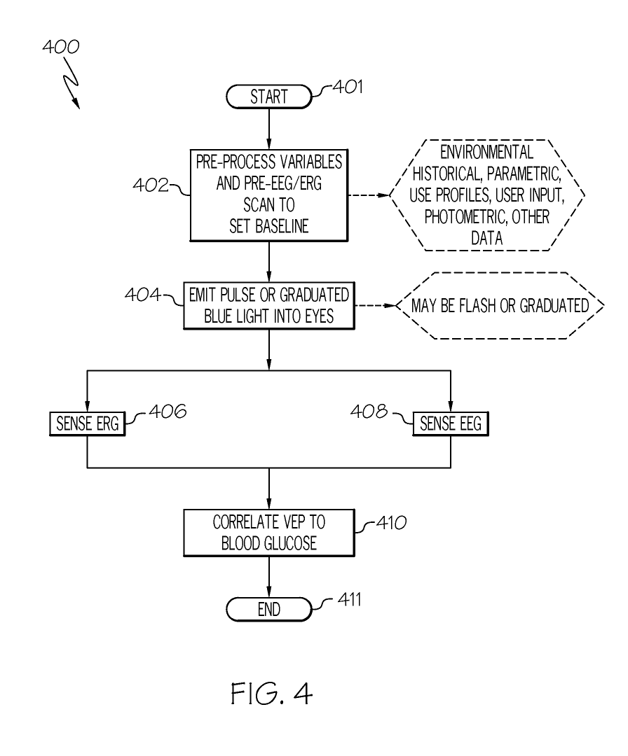

With reference now to FIG. 4, a description of the process 400 for determining blood glucose concentration based on the response of ipGRCs to a single color of light will now be described. Initially, the processor 108 commands a baseline reading of the ERG and/or EEG sensors 104, 106 and pre-processes these readings and, in some embodiments, other pre-process variables to set a baseline (402). These other pre-process variables include, as noted above, various individual baseline data (heart rate and other parametric data), user profiles, historic data, environmental data, sensor data (including photometric), and/or user provided/input data. Thereafter, the processor 108 commands the light source 102 to graduate, pulse or flash blue light 112 into the eye(s) 114 of the user 110 (404). The blue light 112, as noted above, bleaches ipGRCs in the eye(s) 114, causing the ipGRCs to undergo pigment regeneration. During the bleaching and pigment regeneration processes, or the initiation of VEPs, the electrical responses of the eye(s) 114 are sensed by the ERG sensor 104 (406) and/or the neurophysiological brain activity is sensed by the neurophysiological brain activity sensors 106 (408). The processor 108 correlates the sensed electrical responses of the eye(s) 114 and the sensed neurophysiological brain activity to the glucose concentration in the blood of the user 110 (410).

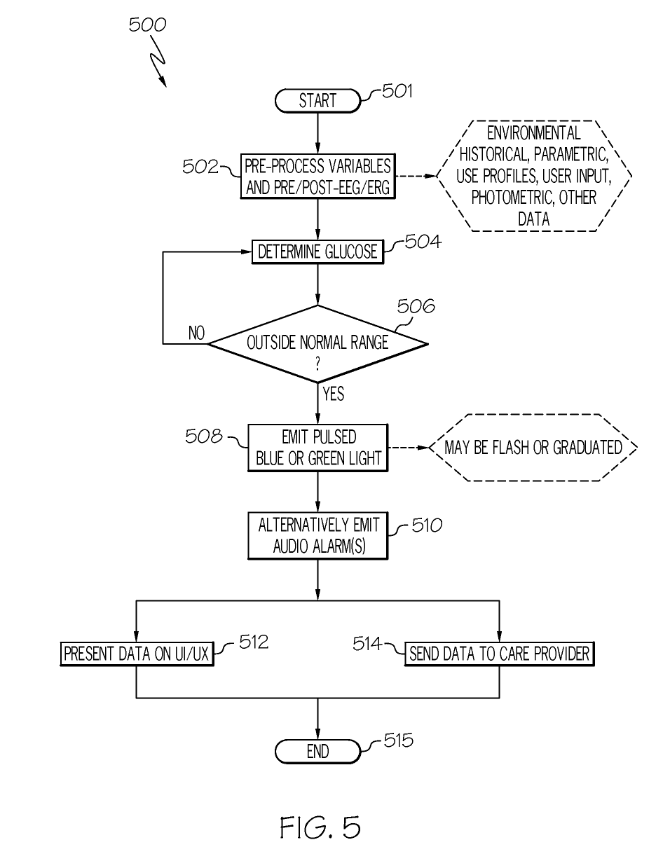

Reference should now be made to FIG. 5, which depicts the process 500 for operation during sleep hours. As before, the processor 108 initially commands a baseline reading of the ERG and EEG sensors 104, 106 and pre-processes these readings and, in some embodiments, other pre-process variables to set a baseline (502). These other pre-process variables include, as noted above, various individual baseline data (heart rate and other parametric data), user profiles, historic data, environmental data, sensor data (including photometric), and/or user provided/input data. Thereafter, the processor commands the light source 102 to emit only red light or red and yellow light, and correlates the sensed electrical responses of the eye(s) 114 and/or the sensed neurophysiological brain activity to the glucose concentration in the blood of the user 110 (504). A determination is then made as to whether the determined glucose level is not outside a predetermined “normal” range (506). If the determined glucose level is not outside the predetermined range, the previous step is repeated. If, however, the determined glucose level is outside the predetermined range, then the processor commands the light source 102 to emit graduated, flashed and/or pulsed blue or green light to wake the user 110 (508). The processor 108 may also generate one or more alerts (510) to assist in waking the user 110. The blood glucose concentration may then be presented on the user interface 145 (512) and/or transmitted to a healthcare provider (514).

The system and method described herein allows measurement of blood glucose concentration from both the visual pigment regeneration of cones/rods or stimulation of electrical potentials from modulation, or in case of ipRGCs the electrical potentials initiated as a function of blood glucose from a pulse, a flash or a graduation of light. The system and method includes color modulation that relies on both gradual bleaching and pulses/flashes of light in cone and rod photoreceptor cells, and additionally includes ipRGCs for blood glucose concentration determination (including in blind or near-blind individuals since it has been found that even blind individuals have EEG response to certain frequencies of light directed at ipRGCs). The system and method are not limited to regeneration of pigment, but includes evaluation of electrical signals related to the initiation of electrical potentials for photoreceptors generally through light stimulation.

Those of skill in the art will appreciate that the various illustrative logical blocks, modules, circuits, and algorithm steps described in connection with the embodiments disclosed herein may be implemented as electronic hardware, computer software, mechanical hardware or combinations thereof. Some of the embodiments and implementations are described above in terms of functional and/or logical block components (or modules) and various processing steps. However, it should be appreciated that such block components (or modules) may be realized by any number of hardware, software, and/or firmware components configured to perform the specified functions. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments described herein are merely exemplary implementations.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal, or in a cloud-based computing platform.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, wirelessly, electronically, logically, or in any other manner, through one or more additional elements.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for determining glucose concentration in the blood of a user, the method comprising the steps of:
emitting light of a first color into an eye of the user;
emitting light of a second color into the eye of the user;
sensing neurophysiological brain activity and electrical responses of the eye of the user during and after emitting the light of the first and second colors into the eye of the user; and
in a processor, correlating one or both of the sensed neurophysiological brain activity and the sensed electrical responses of the eye to the glucose concentration in the blood of the user.

2. The method of claim 1, wherein:
the step of emitting light of a first color comprises varying intensity of the light of the first color for a predetermined time period to attain a predetermined maximum intensity; and
the step of emitting light of a second color comprises varying intensity of the light of the second color for the predetermined time period to attain the predetermined maximum intensity.

3. The method of claim 2, further comprising:
using a camera to take an image of the eye of the user;
evaluating the image, in the processor, to determine retinal health of the eye; and
determining the predetermined time period and the predetermined maximum intensity based on the retinal health.

4. The method of claim 1, wherein:
the step of emitting light of a first color comprises emitting a pulse of light of the first color at a predetermined intensity and for a predetermined time period; and
the step of emitting light of a second color comprises emitting a pulse of light of the second color at the predetermined intensity and for the predetermined time period

5. The method of claim 4, further comprising:
using a camera to take an image of the eye of the user;
evaluating the image, in the processor, to determine retinal health of the eye; and
determining the predetermined time period and the predetermined intensity based on the retinal health.

6. The method of claim 1, wherein the first and second colors are opponent colors.

7. The method of claim 1, further comprising:
controlling the light of the first and second colors to limit retinal pigment bleaching in the eye of the user to a predetermined maximum bleaching percentage.

8. The method of claim 7, wherein the predetermined maximum bleaching percentage is ≦20%.

9. The method of claim 1, wherein:
the light of the first color corresponds with long-preferring photoreceptor cones; and
the light of the second color corresponds with middle-preferring photoreceptor cones.

10. The method of claim 1, wherein:
the light of the first color corresponds with long-preferring photoreceptor rods; and
the light of the second color corresponds with middle-preferring photoreceptor rods.

11. The method of claim 1, further comprising:
generating an alert to remind the user to check blood glucose concentration.

12. The method of claim 1, further comprising:
generating feedback representative of the glucose concentration in the blood of the user.

13. A non-invasive blood glucose concentration sensing system, comprising:
a light source configured to emit light of a first color and light of a second color into an eye of a user;
a neurophysiological brain activity sensor configured to sense neurophysiological brain activity of a user and supply neurophysiological brain activity signals representative thereof;
an electroretinography (ERG) sensor configured to sense electrical responses of the eye of the user; and supply ERG signals representative thereof; and
a processor in operable communication with the light source, the neurophysiological brain activity sensor, and the ERG sensor, the processor coupled to receive the neurophysiological brain activity signals and the ERG signals and configured to:
control the light source to emit the light of the first color and then emit the light of the second color,
process the neurophysiological brain activity signals and the ERG signals, and
correlate one or both of the sensed neurophysiological brain activity and the sensed electrical responses of the eye to the glucose concentration in the blood of the user.

14. The system of claim 13, wherein the processor is configured to control the light source to:
varying intensity of the light of the first color for a predetermined time period to attain a predetermined maximum intensity; and
varying intensity of the light of the second color for the predetermined time period to attain the predetermined maximum intensity.

15. The system of claim 14, further comprising:
a camera configured to take an image of the eye of the user and supply image data thereof to the processor,
wherein the processor is further configured to (i) evaluate the image data to determine retinal health of the eye and (ii) set the predetermined time period and the predetermined maximum intensity based on the retinal health.

16. The system of claim 13, wherein the processor is configured to control the light source to:
emit a pulse of light of the first color at a predetermined intensity and for a predetermined time period; and
emit a pulse of light of the second color at the predetermined intensity and for the predetermined time period

17. The system of claim 16, further comprising:
a camera configured to take an image of the eye of the user and supply image data thereof to the processor,
wherein the processor is further configured to (i) evaluate the image data to determine retinal health of the eye and (ii) set the predetermined time period and the predetermined intensity based on the retinal health.

18. The system of claim 13, wherein the first and second colors are opponent colors.

19. The system of claim 13, wherein the processor is further configured to control the light source to limit retinal pigment bleaching in the eye of the user to a predetermined maximum bleaching percentage.

20. A method for determining glucose concentration in the blood of a user, the method comprising the steps of:
emitting blue light into an eye of the user;
sensing neurophysiological brain activity and electrical responses of the eye of the user during and after emitting the blue light into the eye of the user; and
correlating one or both of the sensed neurophysiological brain activity and the sensed electrical responses of the eye to the glucose concentration in the blood of the user.
